(19) United States
(12) Patent Application Publication
(10) Pub. No.: US 2024/0170734 A1
(43) Pub. Date: May 23, 2024

(54) Flame Retardants for Battery Electrolytes

(71) Applicant: Albemarle Corporation, Charlotte, NC (US)

(72) Inventors: Zhongxin Ge, Baton Rouge, LA (US); Joseph Baker, Baton Rouge, LA (US); Yunqi Liu, Baton Rouge, LA (US); Huaxiang Yang, Sugar Land, TX (US)

(21) Appl. No.: 18/283,992

(22) PCT Filed: May 10, 2022
(86) PCT No.: PCT/US2022/028468

§ 371 Date: Sep. 25, 2023

Related U.S. Application Data
(60) Provisional application No. 63/187,107, filed on May 11, 2021.

(51) Int. Cl. H01M 10/42; H01M 10/0567; H01M 50/383

(52) U.S. Cl. CPC H01M 10/4235; H01M 10/0567; H01M 50/383

(57) ABSTRACT

This invention provides nonaqueous electrolyte solutions for lithium batteries. The nonaqueous electrolyte solutions comprise a liquid electrolyte medium; a lithium-containing salt; and at least one oxygen-containing brominated flame retardant.

### TECHNICAL FIELD

This invention relates to brominated flame retardants for electrolyte solutions for batteries.

### BACKGROUND

One of the components impacting the safety of lithium-ion batteries is their use of flammable solvents in the lithium-containing electrolyte solutions. Inclusion of a flame retardant in the electrolyte solution is one way to mitigate the flammability of these solutions. For a flame retardant to be a suitable component of an electrolyte solution, solubility in the electrolyte is needed, along with electrochemical stability over the range of battery operation, and minimal negative effect on battery performance. Negative effects on battery performance can include reduced conductivity chemical instability to the active material, consumption of lithium, and/or formation of resistive interfaces on the active materials, which can have a deleterious impact on solid electrolyte interface (SEI) formation during initial cycling, resulting in chemical degradation of the electrolyte.

What is desired is a flame retardant that can effectively suppress the flammability of lithium ion batteries with minimal impact to the electrochemical performance of the lithium ion battery at a reasonable cost.

### SUMMARY OF THE INVENTION

This invention provides nonaqueous electrolyte solutions for lithium batteries which contain at least one oxygen-containing brominated flame retardant. In the presence of the oxygen-containing brominated flame retardant(s), fires are extinguished in these nonaqueous electrolyte solutions, at least under laboratory conditions.

An embodiment of this invention is a nonaqueous electrolyte solution for a lithium battery, which solution comprises i) a liquid electrolyte medium; ii) a lithium-containing salt; and iii) at least one oxygen-containing brominated flame retardant selected from
a) a brominated monoester
   α) which has three carbon atoms and at least one bromine atom, or
   (3) which has at least four carbon atoms and at least one bromine atom, and is represented by the formula

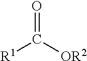

wherein
   R¹ and R² each have at least one carbon atom, and at least one of R¹ and R² has at least one bromine atom, with the proviso that when R² is a benzyl group and there is at least one bromine atom in the benzyl group, R¹ is a branched or straight chain alkyl group; and
b) a brominated diester which has at least six carbon atoms and at least one bromine atom, and is represented by the formula

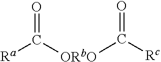

wherein
   R^{a}, R^{b}, and R^{c} each have at least one carbon atom, and at least one of R^{a}, R^{b}, and R^{c} has at least one bromine atom.

Another embodiment of this invention is a nonaqueous electrolyte solution for a lithium battery, which solution comprises i) a liquid electrolyte medium; ii) a lithium-containing salt; and iii) at least one oxygen-containing brominated flame retardant. The oxygen-containing brominated flame retardant is selected from the group consisting of methyl bromoacetate, 2-bromoethyl acetate, 4-bromobutyl acetate, 2-bromovinyl acetate, 3-bromoallyl acetate, 3-bromoallyl butyrate, 2,3-dibromoprop-2-en-1-yl acetate, 2,3-dibromoprop-2-en-1-yl propionate, (3,5-dibromophenyl)methyl acetate, 3-bromo-2,2-bis(bromomethyl)propyl acetate, 3-bromo-2-propenoate, methyl 2-bromo-acrylate, 2,3-dibromoprop-2-en-1-yl bromoacetate, 2,3-dibromoprop-2-en-1-yl 2-methylpropanoate, (3,5-dibromophenyl)methyl bromoacetate, 2,3-dibromobut-2-ene-1,4-diyl diacetate, 2,3-dibromobut-2-ene-1,4-diyl bis(2-methylpropanoate), 2,3-dibromobut-2-ene-1,4-diyl bis(2-bromobutanoate), 2,2-bis(bromomethyl)-1,3-propanediyl diacetate, and 2,2-bis(bromomethyl)propane-1,3-diyl bis(bromoacetate).

These and other embodiments and features of this invention will be still further apparent from the ensuing description and appended claims.

### FURTHER DETAILED DESCRIPTION OF THE INVENTION

Throughout this document, the phrase “electrolyte solution” is used interchangeably with the phrase “nonaqueous electrolyte solution”.

The liquid electrolyte medium is comprised of one or more solvents that typically form the liquid electrolyte medium for lithium electrolyte solutions used in lithium batteries, which solvents are polar and aprotic, stable to electrochemical cycling, and preferably have low viscosity. These solvents usually include noncyclic carbonic acid esters, cyclic carbonic acid esters, ethers, sulfur-containing compounds, and esters of boric acid.

The solvents that can form the liquid electrolyte medium in the practice of this invention include ethylene carbonate (1,3-dioxolan-2-one), dimethyl carbonate, ethyl methyl carbonate, diethyl carbonate, dioxolane, dimethoxy ethane (glyme), tetrahydrofuran, ethylene sulfite, 1,3-propylene glycol boric ester, bis(2,2,2-trifluoroethyl)ether, and mixtures of any two or more of the foregoing.

Preferred solvents include ethylene carbonate, ethyl methyl carbonate, and mixtures thereof. More preferred are mixtures of ethylene carbonate and ethyl methyl carbonate, especially at volume ratios of ethylene carbonate:ethyl methyl carbonate ratios of about 20:80 to about 40:60, more preferably about 25:75 to about 35:65.

Suitable lithium-containing salts in the practice of this invention include lithium perchlorate, lithium nitrate, lithium thiocyanate, lithium aluminate, lithium tetrachloroaluminate, lithium tetrafluoroaluminate, lithium tetraphenylborate, lithium tetrafluoroborate, lithium bis(oxolato)borate (LiBOB), lithium di(fluoro)(oxalato)borate, lithium hexafluorophosphate, lithium hexafluoroarsenate, lithium hexafluoroantimonate, lithium titanium oxide, lithium manganese oxide, lithium cobalt oxide (LiCoO₂), lithium nickel oxide (LiNiO₂), lithium alkyl carbonates in which the alkyl group has 1 to 6 carbon atoms, lithium methyl sulfonate, lithium trifluoromethyl sulfonate, lithium pentafluoroethylsulfonate, lithium pentafluorophenylsulfonate, lithium fluorosulfonate, lithium bis(trifluoromethylsulfonyl)imide, lithium bis(pentafluoroethylsulfonyl)imide, lithium (ethylsulfonyl)(trifluoromethylsulfonyl)imide, and mixtures of any two or more of the foregoing. Preferred lithium-containing salts include lithium hexafluorophosphate, lithium tetrafluoroborate, lithium di(fluoro)(oxolato)borate, and lithium bis(oxolato)borate.

Typical concentrations for the lithium-containing salt in the electrolyte solution are in the range of about 0.1 M to about 2.5 M, preferably about 0.5 M to about 2 M, more preferably about 0.75 M to about 1.75 M, and still more preferably about 0.95 M to about 1.5 M. When more than one lithium-containing salt forms the lithium-containing electrolyte, the concentration refers to the total concentration of all of the lithium-containing salts present in the electrolyte solution.

The electrolyte solution can contain other salts in addition to lithium salts, unless such other salt(s) materially degrade either the performance of the battery for the desired application, or the flame retardancy of the electrolyte solution. Suitable electrolytes other than lithium salts include other alkali metal salts, e.g., sodium salts, potassium salts, rubidium salts, and cesium salts, and alkaline earth metal salts, e.g., magnesium salts, calcium salts, strontium salts, and barium salts. In some aspects, the salts in the non-aqueous electrolyte solution are only one or more lithium salts.

Suitable alkali metal salts that can be present in the electrolyte solution include sodium salts such as sodium chloride, sodium bromide, sodium iodide, sodium perchlorate, sodium nitrate, sodium thiocyanate, sodium aluminate, sodium tetrachloroaluminate, sodium tetrafluoroaluminate, sodium tetraphenylborate, sodium tetrafluoroborate, and sodium hexafluorophosphate; and potassium salts such as potassium chloride, potassium bromide, potassium iodide, potassium perchlorate, potassium nitrate, potassium thiocyanate, potassium aluminate, potassium tetrachloroaluminate, potassium tetrafluoroaluminate, potassium tetraphenylborate, potassium tetrafluoroborate, and potassium hexafluorophosphate.

Suitable alkaline earth metal salts that can be present in the electrolyte solution include magnesium salts such as magnesium chloride, magnesium bromide, magnesium iodide, magnesium perchlorate, magnesium nitrate, magnesium thiocyanate, magnesium aluminate, magnesium tetrachloroaluminate, magnesium tetrafluoroaluminate, magnesium tetraphenylborate, magnesium tetrafluoroborate, and magnesium hexafluorophosphate; and calcium salts such as calcium chloride, calcium bromide, calcium iodide, calcium perchlorate, calcium nitrate, calcium thiocyanate, calcium aluminate, calcium tetrachloroaluminate, calcium tetrafluoroaluminate, calcium tetraphenylborate, calcium tetrafluoroborate, and calcium hexafluorophosphate.

In the practice of this invention, liquid brominated flame retardants are miscible with the liquid medium of the nonaqueous electrolyte solution, where “miscible” means that the brominated flame retardant does not form a separate phase from the electrolyte solution. More specifically, a brominated flame retardant is miscible if it forms a single phase in a mixture of 30 wt % ethylene carbonate and 70 wt % ethyl methyl carbonate which contains 1.2 M lithium hexafluorophosphate, after 24 hours of shaking in a mechanical shaker, and no separate phase is formed after the shaking is stopped, and the brominated flame retardant does not precipitate from, or form a suspension or slurry in, the nonaqueous electrolyte solution. It is recommended and preferred that the brominated flame retardant does not cause the precipitation of, or formation of a suspension or slurry of, any of the other components of the nonaqueous electrolyte solution.

In the practice of this invention, solid brominated flame retardants are soluble in the liquid medium of the nonaqueous electrolyte solution, where “soluble” means that the brominated flame retardant does not precipitate from the electrolyte solution. More specifically, a brominated flame retardant is soluble if it forms a single phase in a mixture of 30 wt % ethylene carbonate and 70 wt % ethyl methyl carbonate which contains 1.2 M lithium hexafluorophosphate, after 24 hours of shaking in a mechanical shaker, and no separate phase or precipitate is formed after the shaking is stopped, and the brominated flame retardant does not precipitate from, or form a suspension or slurry in, the nonaqueous electrolyte solution. It is recommended and preferred that the brominated flame retardant does not cause the precipitation of, or formation of a suspension or slurry of, any of the other components of the nonaqueous electrolyte solution.

In the practice of this invention, the oxygen-containing brominated flame retardants generally have a bromine content of about 30 wt % or more, preferably about 35 wt % or more, based on the weight of the oxygen-containing brominated flame retardant. The oxygen-containing brominated flame retardants in the practice of this invention have a bromine content in the molecule that ranges from about 30 wt % to about 75 wt %, more preferably about 35 wt % to about 75 wt %.

The boiling point of the brominated flame retardants in this invention are about 75° C. or more, preferably about 95° C. or more. Generally, the brominated flame retardants used in the practice of this invention have boiling points near or above the boiling point of the solvent or solvent mixture of the nonaqueous electrolyte solution. The boiling points described throughout this document are at standard temperature and pressure (standard conditions) unless otherwise stated.

The brominated flame retardants used in the practice of this invention are generally polar and aprotic, and stable to electrochemical cycling. Liquid brominated flame retardants preferably also have low viscosities.

In the practice of this invention, a flame retardant amount in the nonaqueous electrolyte solution means enough flame retardant is present that the solution passes the modified horizontal UL-94 test described below. The flame retardant amount is often different for different brominated flame retardants. For the brominated monoesters of the invention, the flame retardant amount in the electrolyte solution is usually about 9.5 wt % or more, preferably about 10 wt % or more of bromine (atoms), relative to the total weight of the nonaqueous electrolyte solution. For the brominated diesters, the flame retardant amount in the electrolyte solution is usually about 9.5 wt % or more, preferably about 11 wt % or more of bromine (atoms), relative to the total weight of the nonaqueous electrolyte solution. For both the brominated monoesters and the brominated diesters of this invention, the flame retardant amount in terms of molecules is usually about 20 wt % or more, sometimes 25 wt % or more, relative to the total weight of the nonaqueous electrolyte solution.

The oxygen-containing brominated flame retardants of this invention share some overall characteristics. In these brominated flame retardants, the bromine content is about 30 wt % or more, preferably about 35 wt % or more, preferably about 30 wt % to about 75 wt %, preferably about 35 wt % to about 72 wt %, relative to the total weight of the flame retardant molecule; and there is at least one bromine atom, preferably one to about six bromine atoms, in the oxygen-containing brominated flame retardant molecule.

In some embodiments, the oxygen-containing brominated flame retardant is a brominated monoester which has at least four carbon atoms, preferably four to about twelve carbon atoms, more preferably four to about ten carbon atoms, and at least one bromine atom, preferably one to about eight bromine atoms, more preferably one to about six bromine atoms, still more preferably one to about four bromine atoms.

When the brominated monoester has four or more carbon atoms, the brominated monoester is represented by the formula

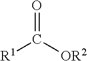

in which R¹ and R² each have at least one carbon atom, and at least one of R¹ and R² has at least one bromine atom. In some embodiments, R¹ contains bromine; in other embodiments, R² contains bromine; and in still other embodiments, both R¹ and R² contain bromine. In some embodiments in which there is only one bromine atom in R¹ and/or R², the bromine atom is located on the terminal (w) carbon atom of the group. In some embodiments, R² does not contain quaternary carbon atoms; in other embodiments, R¹ does not contain quaternary carbon atoms; preferably, neither R¹ nor R² contain quaternary carbon atoms.

In embodiments in which R¹ contains bromine, R¹ preferably has one to about eight carbon atoms, more preferably one to about four carbon atoms, and one to about four bromine atoms, preferably one to about three bromine atoms, more preferably one to about two bromine atoms. The bromine-containing R¹ group can be a branched or straight chain alkyl group, a branched or straight chain alkenyl group, an aryl group, or an ar-alkyl group, and is preferably a branched or straight chain alkenyl group. Preferred alkenyl groups when bromine is present in R¹ include vinyl, allyl, butenyl, pentenyl, and hexenyl, especially vinyl and allyl. In these embodiments, in which R¹ contains bromine and R² does not contain bromine, R² preferably has one to about seven carbon atoms, more preferably one to about three carbon atoms, and can be a branched or straight chain alkyl group, a branched or straight chain alkenyl group, an aryl group, or an ar-alkyl group, and is preferably a branched or straight chain alkyl group. Preferred alkyl groups for R² when it does not contain bromine include methyl, ethyl, n-propyl, and n-butyl; more preferred are methyl and ethyl, especially methyl.

In embodiments in which R² contains bromine, R² preferably has two to about eight carbon atoms, more preferably two to about seven carbon atoms, and one to about four bromine atoms, preferably one to about three bromine atoms. The bromine-containing R² group can be a branched or straight chain alkyl group, a branched or straight chain alkenyl group, an aryl group, or an ar-alkyl group. Preferred groups for R² when bromine is present include methyl, ethyl, n-propyl, iso-propyl, n-butyl, isobutyl, sec-butyl, neopentyl, vinyl, allyl, butenyl, pentenyl, hexenyl, and benzyl; more preferred are ethyl, n-butyl, neopentyl, vinyl, allyl, and benzyl. In embodiments in which R² contains bromine and R¹ does not contain bromine, le preferably has one to about eight carbon atoms, more preferably one to about six carbon atoms, and can be a branched or straight chain alkyl group, a branched or straight chain alkenyl group, an aryl group, or an ar-alkyl group, and is preferably a branched or straight chain alkyl group. Preferred alkyl groups for R¹ when it does not contain bromine include methyl, ethyl, n-propyl, isobutyl, and n-butyl; more preferred are methyl, ethyl, n-propyl, and isopropyl, especially methyl.

When R² is a benzyl group and there is at least one bromine atom in the benzyl group, R¹ is a branched or straight chain alkyl group, and optionally and preferably, R¹ contains one or more bromine atoms.

In embodiments in which both R¹ and R² contain at least one bromine atom, le preferably has one to about eight carbon atoms, more preferably one to about four carbon atoms, and one to about four bromine atoms, preferably one to about three bromine atoms, more preferably one to about two bromine atoms. R¹ can be a branched or straight chain alkyl group, a branched or straight chain alkenyl group, an aryl group, or an ar-alkyl group, and is preferably a branched or straight chain alkyl group. Preferred alkyl groups include methyl, ethyl, n-propyl, and n-butyl; more preferred are methyl and ethyl, especially methyl. When both R¹ and R² contain bromine, a preferred bromine-containing R¹ group is bromomethyl.

In these embodiments in which both R¹ and R² contain bromine, R² has one to about nine carbon atoms, more preferably about two to about eight carbon atoms, and one to about four bromine atoms, preferably one to about three bromine atoms, more preferably one to about two bromine atoms. R² can be a branched or straight chain alkyl group, a branched or straight chain alkenyl group, an aryl group, or an ar-alkyl group, and is preferably a branched or straight chain alkenyl group or an ar-alkyl group. Preferred groups for R² include methyl, ethyl, n-propyl, iso-propyl, n-butyl, isobutyl, sec-butyl, vinyl, allyl, butenyl, pentenyl, hexenyl, and benzyl; more preferred are allyl and benzyl. When both R¹ and R² contain bromine, preferred bromine-containing R² groups include 2,3-dibromoallyl, (3,5-dibromophenyl)methyl, and (4-bromophenyl)methyl.

Preferably, the brominated monoester is methyl bromoacetate, 2-bromoethyl acetate; 4-bromobutyl acetate; 2-bromovinyl acetate; 3-bromoallyl acetate; 3-bromoallyl butyrate; 2,3-dibromoprop-2-en-1-yl acetate, also called 2,3-dibromoallyl acetate; 2,3-dibromoprop-2-en-1-yl propanoate, also called 2,3-dibromoallyl propanoate; 2,3-dibromoprop-2-en-1-yl 2-methylpropanoate, also called 2,3-dibromoprop-2-en-1-yl isobutyrate; (3,5-dibromophenyl)methyl acetate; 3-bromo-2,2-bis(bromomethyl)propyl acetate, also called tribromoneopentyl acetate; methyl 3-bromo-2-propenoate; methyl 2-bromo-acrylate; 2,3-dibromoprop-2-en-1-yl bromoacetate; or (3,5-dibromophenyl)methyl bromoacetate.

In some embodiments, the brominated monoester has three carbon atoms and at least one bromine atom, preferably one to about four bromine atoms, more preferably one to about three bromine atoms. Brominated monoesters having three carbon atoms can be represented by the formula shown above; as described above, in some embodiments, R¹ contains bromine; in other embodiments, R² contains bromine; and in still other embodiments, both R¹ and R² contain bromine. In some preferred embodiments, R¹ is bromomethyl. Preferred brominated monoesters having three carbon atoms include methyl bromoacetate.

In the practice of this invention, the brominated monoesters preferably have four or more carbon atoms.

In another embodiment, the oxygen-containing brominated flame retardant is a brominated diester which has one to about eight bromine atoms, preferably one to about six bromine atoms, more preferably two to about six bromine atoms. There are about six to about twenty carbon atoms, preferably about six to about eighteen carbon atoms, more preferably about six to about fifteen carbon atoms in the brominated diester. The bromine content of the brominated diesters is about 35 wt % or more, preferably about 35 wt % to about 70 wt %, more preferably about 40 wt % to about 65 wt %, based on the weight of the brominated diester. The brominated diester is represented by the formula

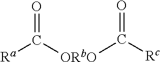

wherein R^{a}, R^{b}, and R^{c} each have at least one carbon atom, and at least one of R^{a}, R^{b}, and R^{c} has at least one bromine atom. Preferably, R^{b} has at least one bromine atom.

R^{a} and R^{c} each, independently, have one to about six carbon atoms, preferably one to about five carbon atoms, more preferably one to about four carbon atoms, and one to about four bromine atoms, preferably one to about three bromine atoms, more preferably one to about two bromine atoms. Each of R^{a} and R^{c}, independently, can be a branched or straight chain alkyl group, a branched or straight chain alkenyl group, or an aryl group. Preferred alkyl groups for R^{a} and R^{c} include methyl, ethyl, n-propyl, isopropyl, and n-butyl; more preferred are methyl, n-propyl, and isopropyl; preferred alkenyl groups include vinyl, allyl, butenyl, pentenyl, and hexenyl; preferred aryl groups include phenyl. In some embodiments, R^{a} and/or R^{c} may not contain bromine. R^{a} and R^{c} can be the same or different; in some embodiments, R^{a} and R^{c} are preferably the same.

R^{b} is the group bridging the two ester moieties, and R^{b} preferably has two to about eight carbon atoms, preferably two to about six carbon atoms, and one to about four bromine atoms, preferably one to about three bromine atoms, more preferably one to about two bromine atoms. Preferably, at least two carbon atoms of R^{b} form the bridge between the two ester moieties. R^{b} is preferably a group other than a phenylene or arylene group. R^{b} can be a branched or straight chain alkylenyl group, or a branched or straight chain alkenylenyl group. In some preferred embodiments, R^{b} does not contain quaternary carbon atoms. For R^{b} , preferred alkyl groups include ethyl, n-propyl, n-butyl, and neopentyl; preferred alkenyl groups include ethylenyl, propylenyl, butenyl, pentenyl, and hexenyl, especially butenyl.

In some embodiments in which bromine atoms are present in R^{b}, groups R^{a} and R^{c} do not contain bromine atoms. In other embodiments, bromine atoms are present in R^{a} and/or R^{c}, preferably, at least one bromine atom is present in each of R^{a}, R^{b}, and R^{c}.

When R^{b} contains a quaternary carbon atom, there is preferably at least one bromine atom present in each of R^{a}, R^{b}, and R^{c}. More preferably, there is at least one bromine atom present in R^{b} and groups R^{a} and R^{c} do not contain bromine atoms. Preferably, neither R^{a} nor R^{b} contain quaternary carbon atoms.

Preferred brominated diesters include 2,3-dibromobut-2-ene-1,4-diyl diacetate; 2,3-dibromobut-2-ene-1,4-diyl bis(2-methylpropanoate), also called 2,3-dibromobutene-1,4-diyl-1,4-diisobutyrate; 2,3-dibromobut-2-ene-1,4-diyl bis(2-bromobutanoate); 2,2-bis(bromomethyl)-1,3-propanediyl diacetate, also called dibromoneopentyl diacetate; and 2,2-bis(bromomethyl)propane-1,3-diyl bis(bromoacetate).

In some preferred embodiments of the invention, the liquid electrolyte medium is ethylene carbonate, ethyl methyl carbonate, or a mixture thereof. More preferably, the lithium-containing salt is lithium hexafluorophosphate, lithium di(fluoro)(oxalato)borate, or lithium bis(oxalato)borate.

In some embodiments of the invention, at least one electrochemical additive is included in the nonaqueous electrolyte solution.

In the practice of this invention, the electrochemical additives are soluble in, or miscible with, the liquid medium of the nonaqueous electrolyte solution. Electrochemical additives that are in liquid form are miscible with the liquid medium of the nonaqueous electrolyte solution, where “miscible” means that the electrochemical additives do not form a separate phase from the electrolyte solution. More specifically, an electrochemical additive is miscible if it forms a single phase in a mixture of 30 wt % ethylene carbonate and 70 wt % ethyl methyl carbonate which contains 1.2 M lithium hexafluorophosphate, after 24 hours of shaking in a mechanical shaker, and no separate phase is formed after the shaking is stopped, and the electrochemical additive does not precipitate from, or form a suspension or slurry in, the nonaqueous electrolyte solution.

The term “soluble,” usually used for electrochemical additives in solid form, indicates that, once dissolved, the electrochemical additive does not precipitate from, or form a suspension or slurry in, the nonaqueous electrolyte solution. More specifically, an electrochemical additive is soluble if it dissolves in a mixture of 30 wt % ethylene carbonate and 70 wt % ethyl methyl carbonate which contains 1.2 M lithium hexafluorophosphate, after 24 hours of shaking in a mechanical shaker, if no precipitate, suspension, or slurry is formed after the shaking is stopped. It is recommended and preferred that the electrochemical additive does not cause the precipitation of, or formation of a suspension or slurry of, any of the other components of the nonaqueous electrolyte solution.

The brominated flame retardant, electrochemical additive, and mixtures thereof are generally stable to electrochemical cycling, and preferably have low viscosities and/or do not significantly increase the viscosity of the nonaqueous electrolyte solution.

In various embodiments, the electrochemical additive is selected from a) unsaturated cyclic carbonates containing three to about four carbon atoms, b) fluorine-containing saturated cyclic carbonates containing three to about four carbon atoms and one to about two fluorine atoms, c) tris(trihydrocarbylsilyl) phosphites containing three to about six carbon atoms, d) trihydrocarbyl phosphates containing three to about nine carbon atoms, e) cyclic sultones containing three to about four carbon atoms, f) saturated cyclic hydrocarbyl sulfites having a 5-membered ring and containing two to about four carbon atoms, g) saturated cyclic hydrocarbyl sulfates having a 5-membered ring and containing two to about four carbon atoms, h) cyclic dioxadithio polyoxide compounds having a 6-membered or 7-membered ring and containing two to about four carbon atoms, i) another lithium-containing salt, and j) mixtures of any two or more of the foregoing.

In some embodiments, the electrochemical additive is an unsaturated cyclic carbonate containing three to about six carbon atoms, preferably three to about four carbon atoms. Suitable unsaturated cyclic carbonates include vinylene carbonate (1,3-dioxol-2-one), 4-methyl-1,3-dioxol-2-one, and 4,5-dimethyl-1,3-dioxol-2-one; vinylene carbonate is a preferred unsaturated cyclic carbonate. The unsaturated cyclic carbonate is preferably in an amount of about 0.5 wt % to about 12 wt %, more preferably about 0.5 wt % to about 3 wt % or about 8 wt % to about 11 wt %, relative to the total weight of the nonaqueous electrolyte solution.

When the electrochemical additive is a fluorine-containing saturated cyclic carbonate containing three to about five carbon atoms, preferably three to about four carbon atoms, and one to about four fluorine atoms, preferably one to about two fluorine atoms, suitable fluorine-containing saturated cyclic carbonates include 4-fluoro-ethylene carbonate and 4,5-difluoro-ethylene carbonate. Preferably the fluorine-containing saturated cyclic carbonate is 4-fluoro-ethylene carbonate. The fluorine-containing saturated cyclic carbonate is preferably in an amount of about 0.5 wt % to about 8 wt %, more preferably about 1.5 wt % to about 5 wt %, relative to the total weight of the nonaqueous electrolyte solution.

The tris(trihydrocarbylsilyl) phosphite electrochemical additives contain three to about nine carbon atoms, preferably about three to about six carbon atoms; the trihydrocarbylsilyl groups may be the same or different. Suitable tris(trihydrocarbylsilyl) phosphites include tris(trimethylsilyl) phosphite, bis(trimethylsilyl)(triethylsilyl) phosphite, tris(triethylsilyl) phosphite, bis(trimethylsilyl)(triethyl silyl) phosphite, bis(trimethylsilyl)(tri-n-propylsilyl)phosphite, and tris(tri-n-propylsilyl) phosphite; tris(trimethylsilyl) phosphite is a preferred tris(trihydrocarbylsilyl) phosphite. The tris(trihydrocarbylsilyl) phosphite is preferably in an amount of about 0.1 wt % to about 5 wt %, more preferably about 0.15 wt % to about 4 wt %, even more preferably about 0.2 wt % to about 3 wt %, relative to the total weight of the nonaqueous electrolyte solution.

In some embodiments, the electrochemical additive is a trihydrocarbyl phosphate containing three to about twelve carbon atoms, preferably three to about nine carbon atoms. The hydrocarbyl groups can be saturated or unsaturated, and the hydrocarbyl groups in the trihydrocarbyl phosphate may be the same or different. Suitable trihydrocarbyl phosphates include trimethyl phosphate, triethyl phosphate, dimethyl ethyl phosphate, tri-n-propyl phosphate, triallyl phosphate, and trivinyl phosphate; triallyl phosphate is a preferred trihydrocarbyl phosphate. The trihydrocarbyl phosphate is usually in an amount of about 0.5 wt % to about 5 wt %, preferably about 1 wt % to about 5 wt %, more preferably about 2 wt % to about 4 wt %, relative to the total weight of the nonaqueous electrolyte solution.

When the electrochemical additive is a cyclic sultone containing three to about eight carbon atoms, preferably three to about four carbon atoms, suitable cyclic sultones include 1-propane-1,3-sultone (1,3-propane sultone), 1-propene-1,2-sultone (1,3-propene sultone), 1,3-butane sultone (5-methyl-1,2-oxathiolane 2,2-dioxide), 2,4-butane sultone (3-methyl-1,2-oxathiolane 2,2-dioxide), 1,4-butane sultone (1,2-oxathiane 2,2-dioxide), 2-hydroxy-alpha-toluenesulfonic acid sultone (3H-1,2-benzoxathiole 2,2-dioxide), and 1,8-naphthosultone; preferred cyclic sultones include 1-propane-1,3-sultone and 1-propene-1,3-sultone. The cyclic sultone is preferably in an amount of about 0.25 wt % to about 5 wt %, more preferably about 0.5 wt % to about 4 wt %, relative to the total weight of the nonaqueous electrolyte solution.

The saturated cyclic hydrocarbyl sulfite electrochemical additive contains two to about six carbon atoms, preferably two to about four carbon atoms, and has a 5-membered or 6-membered ring, preferably a 5-membered ring. One or more substituents can be present on the ring, such as methyl or ethyl groups, preferably one or more methyl groups, more preferably, no substituents are present on the ring. Suitable saturated cyclic hydrocarbyl sulfites include 1,3,2-dioxathiolane, 2-oxide (1,2-ethylene sulfite), 1,2-propanediol sulfite (1,2-propylene sulfite), 4,5-dimethyl-1,3,2-dioxathiolane 2-oxide, 1,3,2-dioxathiane 2-oxide, 4-methyl-1,3-dioxathiane, 2-oxide (1,3-butylene sulfite); preferred cyclic hydrocarbyl sulfites include 1,3,2-dioxathiolane, 2-oxide (1,2-ethylene sulfite). The cyclic hydrocarbyl sulfite is preferably in an amount of about 0.5 wt % to about 5 wt %, more preferably about 1 wt % to about 4 wt %, relative to the total weight of the nonaqueous electrolyte solution.

In some embodiments, the electrochemical additive is a saturated cyclic hydrocarbyl sulfate containing two to about six carbon atoms, preferably two to about four carbon atoms, and has a 5-membered or 6-membered ring, preferably a 5-membered ring. One or more substituents can be present on the ring, such as methyl or ethyl groups, preferably one or more methyl groups, more preferably, no substituents are present on the ring. Suitable saturated cyclic hydrocarbyl sulfates include 1,3,2-dioxathiolane 2,2-dioxide (1,2-ethylene sulfate), 1,3,2-dioxathiane 2,2-dioxide (1,3-propylene sulfate), 4-methyl-1,3,2-dioxathiane 2,2-dioxide (1,3-butylene sulfate), and 5,5-dimethyl-1,3,2-dioxathiane 2,2-dioxide. The saturated cyclic hydrocarbyl sulfate is preferably in an amount of about 0.25 wt % to about 5 wt %, more preferably about 1 wt % to about 4 wt %, relative to the total weight of the nonaqueous electrolyte solution.

When the electrochemical additive is a cyclic dioxadithio polyoxide compound, the cyclic dioxadithio polyoxide compound contains two to about six carbon atoms, preferably two to about four carbon atoms, and has 6-membered, 7-membered, or 8-membered ring. Preferably, the cyclic dioxadithio polyoxide compound contains two to about four carbon atoms, and has 6-membered or 7-membered ring. One or more substituents can be present on the ring, such as methyl or ethyl groups, preferably one or more methyl groups, more preferably, no substituents are present on the ring. Suitable cyclic dioxadithio polyoxide compounds include 1,5,2,4-dioxadithiane 2,2,4,4-tetroxide, 1,5,2,4-dioxadithiepane 2,2,4,4-tetraoxide (cyclodisone), 3-methyl-1,5,2,4-dioxadithiepane, 2,2,4,4-tetraoxide, and 1,5,2,4-dioxadithiocane, 2,2,4,4-tetraoxide; 1,5,2,4-dioxadithiane 2,2,4,4-tetroxide is preferred. The cyclic dioxadithio polyoxide compound is preferably in an amount of about 0.5 wt % to about 5 wt %, more preferably about 1 wt % to about 4 wt %, relative to the total weight of the nonaqueous electrolyte solution.

The phrases “another lithium-containing salt” and “other lithium containing salt” indicate that there are at least two lithium salts used in the preparation of the electrolyte solution. When the electrochemical additive is another lithium-containing salt, it is preferably in an amount of about 0.5 wt % to about 5 wt % relative to the total weight of the nonaqueous electrolyte solution. Suitable lithium-containing salts include all of the lithium-containing salts listed above; lithium di(fluoro)(oxolato)borate and lithium bis(oxolato)borate are preferred.

Mixtures of any two or more of the foregoing electrochemical additives can be used, including different electrochemical additives of the same type and/or electrochemical additives of different types. When mixtures of electrochemical additives are used, the combined amount of the electrochemical additives is about 0.25 wt % to about 5 wt % relative to the total weight of the nonaqueous electrolyte solution. Mixtures of an unsaturated cyclic carbonate and a saturated cyclic hydrocarbyl sulfite or mixtures of a cyclic sultone, a tris(trihydrocarbylsilyl) phosphite, and a cyclic dioxadithio polyoxide compound are preferred.

Additional ingredients that are often included in electrolyte solutions for lithium batteries can also be present in the electrolyte solutions of the present invention. Such additional ingredients include nitrile compounds such as succinonitrile and perfluoralkyl nitriles, and silazane compounds such as hexamethyldisilazane. A preferred additional ingredient is a nitrile compound; succinonitrile is a preferred nitrile compound. Typically, the amount of an optional ingredient is in the range of about 1 wt % to about 5 wt %, preferably about 1 wt % to about 4 wt %, relative to the total weight of the nonaqueous electrolyte solution.

In some preferred embodiments, a nitrile compound and another lithium-containing salt are components of the electrolyte solution. Nitrile compounds and lithium-containing salts are as described above. Preferably, the nitrile compound is succinonitrile, and the other lithium-containing salt is preferably lithium di(fluoro)(oxalato)borate.

Another embodiment of this invention provides a process for producing a nonaqueous electrolyte solution for a lithium battery. The process comprises combining components comprising i) a liquid electrolyte medium; ii) a lithium-containing salt; and iii) at least one oxygen-containing brominated flame retardant selected from
a) a brominated monoester
   α) which has three carbon atoms and at least one bromine atom, or
   β) which has at least four carbon atoms and at least one bromine atom, and is represented by the formula

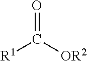

wherein
   R¹ and R² each have at least one carbon atom, and at least one of R¹ and R² has at least one bromine atom, with the proviso that when R² is a benzyl group and there is at least one bromine atom in the benzyl group, R¹ is a branched or straight chain alkyl group; and
b) a brominated diester which has at least six carbon atoms and at least one bromine atom, and is represented by the formula

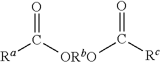

wherein
   R^{a}, R^{b}, and R^{c} each have at least one carbon atom, and at least one of R^{a}, R^{b}, and R^{c} has at least one bromine atom.

Optionally, the components further comprise iv) at least one electrochemical additive as described above. The oxygen-containing brominated flame retardant is present in the electrolyte solution in a flame retardant amount. The ingredients can be combined in any order, although it is preferable to add all of the components to the liquid electrolyte medium. Optional ingredients are also preferably added to the liquid electrolyte medium. Features of, and preferences for, the liquid electrolyte medium, lithium-containing salt, oxygen-containing brominated flame retardant(s), electrochemical additive(s), and amounts of each component, are as described above.

Still another embodiment of this invention provides a process for producing a nonaqueous electrolyte solution for a lithium battery. The process comprises combining components comprising i) a liquid electrolyte medium; ii) a lithium-containing salt; and iii) at least one oxygen-containing brominated flame retardant. Optionally, the components further comprise iv) at least one electrochemical additive as described above. The oxygen-containing brominated flame retardant is selected from the group consisting of methyl bromoacetate, 2-bromoethyl acetate, 4-bromobutyl acetate, 2-bromovinyl acetate, 3-bromoallyl acetate, 3-bromoallyl butyrate, 2,3-dibromoprop-2-en-1-yl acetate, 2,3-dibromoprop-2-en-1-yl propionate, 2,3-dibromoprop-2-en-1-yl 2-methylpropanoate, (3,5-dibromophenyl)methyl acetate, 3-bromo-2,2-bis(bromomethyl)propyl acetate, methyl 3-bromo-2-propenoate, methyl 2-bromo-acrylate, 2,3-dibromoprop-2-en-1-yl bromoacetate, (3,5-dibromophenyl)methyl bromoacetate, 2,3-dibromobut-2-ene-1,4-diyl diacetate, 2,3-dibromobut-2-ene-1,4-diyl bis(2-methylpropanoate), 2,3-dibromobut-2-ene-1,4-diyl bis(2-bromobutanoate), 2,2-bis(bromomethyl)-1,3-propanediyl diacetate, and 2,2-bis(bromomethyl)propane-1,3-diyl bis(bromoacetate). Preferences for the liquid electrolyte medium, lithium-containing salt, electrochemical additive(s), and amounts of each component, are as described above.

The nonaqueous electrolyte solutions of the present invention, which contain one or more brominated flame retardants, are typically used in nonaqueous lithium batteries comprising a positive electrode, a negative electrode, and the nonaqueous electrolyte solution. A nonaqueous lithium battery can be obtained by injecting a nonaqueous electrolyte solution between the negative electrode and the positive electrode optionally having a separator therebetween.

The molecules 2,3-dibromoprop-2-en-1-yl propanoate, 2,3-dibromoprop-2-en-1-yl 2-methylpropanoate, 2,3-dibromoprop-2-en-1-yl bromoacetate, 2,3-dibromobut-2-ene-1,4-diyl bis(2-methylpropanoate), 2,3-dibromobut-2-ene-1,4-diyl bis(2-bromobutanoate), and 2,2-bis(bromomethyl)propane-1,3-diyl bis(bromoacetate) are new compositions of matter.

The following examples are presented for purposes of illustration, and are not intended to impose limitations on the scope of this invention.

In Example 1, a modified horizontal UL-94 test was performed. This modified horizontal UL-94 test is quite similar to known, published horizontal UL-94 tests. See in this regard, e.g., Otsuki, M. et al. “Flame-Retardant Additives for Lithium-Ion Batteries.” Lithium-Ion Batteries. Ed. M. Yoshio et al. New York, Springer, 2009, 275-289. The modified UL-94 test was as follows:
Wicks were cut from round fiberglass wick, and cut edges were made smooth, and then dust and particles were removed from the wick surface. The wicks were dried for 20 hours at 120° C. prior to testing. Wicks were 5±0.1 inch (12.7±0.25 cm) long.
Each specimen to be tested was prepared in a dry box in a 4 oz. (120 mL) glass jar, by combining the desired amount of flame retardant and, when present, electrochemical additive, with the desired amount of the electrolyte solution, e.g., 20 wt % of the brominated flame retardant and 80 wt % of the electrolyte solution were combined to form the electrolyte solution containing the flame retardant. Prior to combination with the flame retardant, the electrolyte solution contained 1.2M LiPF₆ in ethylene carbonate/ethyl methyl carbonate (wt ratio 3:7). Each wick was soaked in the electrolyte solution for 30 minutes.
Each specimen was removed from the electrolyte solution and held over the electrolyte solution until no dripping occurred, and then placed in a 4 oz. (120 ml) glass jar; the cap was closed to prevent electrolyte solution from evaporating.
The burner was ignited and adjusted to produce a blue flame 20±1 mm high.
A specimen was removed from its 4 oz. (120 mL) glass jar, and the specimen was placed on a metal support fixture in a horizontal position, secured at one end of the wick.
If an exhaust fan was running, it was shut off for the test.
The flame was at an angle of 45±2 degrees to the horizontal wick. One way to accomplish this when the burner had a burner tube was to incline the central axis of the burner tube toward an end of the specimen at an angle of 45±2 degrees from the horizontal.
The flame was applied to the free end of the specimen for 30±1 seconds without Changing its position; the burner was removed after 30±1 seconds, or as soon as the combustion front on the specimen reached the 1 inch (2.54 cm) mark.
If the specimen continued to burn after removal of the test flame, the time in seconds was recorded, for either the flame to extinguish or for the combustion front (flame) to travel from the 1 inch (2.54 cm) mark to the 4 inch (10.16 cm) mark.

A specimen was considered to be “not flammable” if the flame extinguished when the burner was removed. A specimen was considered to be “flame retardant” if the flame extinguished before reaching the 1 inch (2.54 cm) mark. A specimen was considered to be “self-extinguishing” if the flame went out before reaching the 4 inch (10.16 cm) mark.

Each modified horizontal UL-94 test result reported below is the average of three runs.

### EXAMPLE 1

Various nonaqueous electrolyte solutions containing different oxygen-containing brominated flame retardants, prepared as described above, were subjected to the modified UL-94 test described above. Results are summarized in Table 1 below; as noted above, the reported numbers are an average value from three runs.

TABLE 1

| | Flame | Bro- | | |
|---|---|---|---|---|
| | re- | mine | | Time |
| | tardant | wt % | | to |
| | wt % | in | | extin- |
| Flame retardant | in soln. | soln. | Result | guish |
| 2-bromoethyl acetate | 20 | 9.58 | flame retardant | 35 s |
| 4-bromobutyl acetate | 25 | 10.2 | flame retardant | 31 s |
| 2,3-dibromoprop-2-en-1-yl | 20 | 12.4 | flame retardant | na¹ |
| acetate | | | | |
| 2,3-dibromoprop-2-en-1-yl 2- | 20 | 11.1 | flame retardant | 28 s |
| methylpropanoate | | | | |
| 2,3-dibromoprop-2-en-1-yl | 20 | 14.2 | flame retardant | 12 s |
| bromoacetate | | | | |
| 2,3-dibromobut-2-ene-1,4-diyl | 20 | 8.3 | fail² | — |
| bis(2-methylpropanoate) | | | | |
| 2,3-dibromobut-2-ene-1,4-diyl | 30 | 12.4 | flame retardant | 16 s |
| bis(2-methylpropanoate) | | | | |
| 2,2-bis(bromomethyl)-1,3- | 25 | 11.6 | flame retardant | 35 s |
| propanediyl diacetate³ | | | | |

¹Value not available.
²Comparative run.
³Over a few weeks, small amounts of solids were formed.

### EXAMPLE 2

Tests of some flame retardants in coin cells were also carried out. Coin cells were assembled using nonaqueous electrolyte solutions containing the desired amount of flame retardant. The coin cells were then subjected to electrochemical cycling of CCCV charging to 4.2 V at C/5, with a current cutoff of C/50 in the CV portion, and CC discharge at C/5 to 3.0 V.

One sample was a nonaqueous electrolyte solution without a flame retardant, and contained 1.2 M LiPF₆ in ethylene carbonate/ethyl methyl carbonate (wt ratio 3:7). The rest of the samples contained the desired amount of flame retardant in the electrolyte solution. Results are summarized in Table 2 below; the error range in the Coulombic efficiencies is about ±0.5% to about ±1.0%.

TABLE 2

| | | | Coulombic | |
|---|---|---|---|---|
| | Flame | Bromine | efficiency | |
| | retardant | in | 1st | 10th |
| Chemical Name | in soln. | soln. | cycle | cycle |
| Electrolyte soln.¹ | 0 | 0 | 81.8% | 99.6% |
| 2-bromoethyl acetate² | 8 wt % | 3.83 wt % | 39.7% | 99.3% |
| 4-bromobutyl acetate² | 8 wt % | 3.28 wt % | 24.1% | 87.5% |
| Methyl bromoacetate² | 8 wt % | 4.18 wt % | 3.77% | 35.4% |

¹Comparative run.
²Data is from single best-performing cell.

### EXAMPLE 3

Synthesis of 2,3-dibromoprop-2-en-1-yl Propanoate

Into a 500 mL 4-neck round bottom flask were charged 2,3-dibromoprop-2-en-1-ol (35.5 g), propionic acid (34.71 g), p-toluenesulfonic acid monohydrate (1.30 g) and toluene (250 mL). The mixture was heated to reflux under agitation and kept at 113 to 117° C. for 5 hours, during which water was removed and collected via a Dean-Stark apparatus. The reaction was monitored by gas chromatography (GC). After reaction was complete as indicated by GC, the reaction mixture was cooled to room temperature, washed several times with saturated aqueous sodium bicarbonate (4×90 mL) and phase separated. The organic layer was dried over MgSO₄ and then filtered. After filtration, toluene was removed from the organic layer using a rotary evaporator, and then the organic layer was further dried under high vacuum (˜1 torr) to obtain 40.56 g product as a clear liquid.

### EXAMPLE 4

Synthesis of 2,3-dibromoprop-2-en-1-yl Bromoacetate

Into a 500 mL 4-neck round bottom flask were charged 2,3-dibromoprop-2-en-1-ol (17.2 g), bromoacetic acid (30 g), p-toluenesulfonic acid monohydrate (0.83 g) and toluene (250 mL).
The mixture was heated to reflux under agitation and kept at 115 to 117° C. for 3 hours, during which water was removed and collected via a Dean-Stark apparatus. The reaction was monitored by gas chromatography (GC). After reaction was complete as indicated by GC, the reaction mixture was cooled to room temperature, washed several times with saturated aqueous sodium bicarbonate (4×80 mL) and phase separated. The organic layer was dried over MgSO₄ and then filtered. After filtration, toluene was removed from the organic layer using a rotary evaporator, and then the organic layer was further dried under high vacuum (0 Torr at 55° C. for 1.5 hours) to obtain 24.97 g of product as a clear liquid.

### EXAMPLE 5

Synthesis of 2,3-dibromoprop-2-en-1-yl 2-methylpropanoate

Into a 500 mL, 4-neck round bottom flask were charged 2,3-dibromobut-2-en-1-ol (34.47 g), isobutyric acid (39.65 g), p-toluenesulfonic acid monohydrate (1.30 g) and toluene (250 mL). The mixture was heated to reflux under agitation and kept at 116 to 118° C. for 4 hours, during which water was removed and collected via a Dean-Stark apparatus. The reaction was monitored by gas chromatography (GC). After reaction was complete as indicated by GC, the reaction mixture was cooled to room temperature, washed several times with saturated aqueous sodium bicarbonate (5×80 mL), and phase separated. The organic layer was dried over MgSO₄ and then filtered. After filtration, toluene was removed from the organic layer using a rotary evaporator, and then the organic layer was further dried under high vacuum (˜1 torr) to obtain 41.12 g of product as a clear liquid.

### EXAMPLE 6

Synthesis of 2,3-dibromobut-2-ene-1,4-diyl bis(2-methylpropanoate)

Into a 500 mL, 4-neck round bottom flask were charged 2,3-dibromobut-2-ene-1,4-diol (49.18 g), isobutyric acid (105.60 g), p-toluenesulfonic acid monohydrate (3.60 g) and toluene (250 mL). The mixture was heated to reflux under agitation and kept at 118° C. for 2 hours, during which water was removed and collected via a Dean-Stark apparatus. The reaction was monitored by gas chromatography (GC). After reaction was complete as indicated by GC, the reaction mixture was cooled to room temperature, washed several times with saturated aqueous sodium bicarbonate (8×80 mL), and phase separated. The organic layer was dried over MgSO₄ and then filtered. After filtration, toluene was removed from the organic layer using a rotary evaporator, and then the organic layer was further dried under high vacuum (˜1 torr) to obtain 73.77 g of product as a clear liquid.

### EXAMPLE 7

Synthesis of 2,3-dibromobut-2-ene-1,4-diyl bis(2-bromobutanoate)

Into a 500 mL, 4-neck round bottom flask were charged 2,3-dibromobut-2-ene-1,4-diol (20 g), 2-bromobutyric acid (54.33 g), p-toluenesulfonic acid monohydrate (1.32 g) and toluene (250 mL). The mixture was heated to reflux under agitation and kept at 117° C. for 4 hours, during which water was removed and collected via a Dean-Stark apparatus. The reaction was monitored by gas chromatography (GC). After reaction was complete as indicated by GC, the reaction mixture was cooled to room temperature, washed several times with saturated aqueous sodium bicarbonate (5×150 mL) and phase separated. The organic layer was dried over MgSO₄ and filtered. After filtration, toluene was removed from the organic layer using a rotary evaporator, and then the organic layer was further dried under high vacuum (˜1 torr) to obtain 37.69 g of product as a slightly yellowish liquid.

### EXAMPLE 8

Synthesis of 2,2-bis(bromomethyppropane-1,3-diyl bis(bromoacetate)

Into a 500 mL, 4-neck round bottom flask were charged 2,2-bis(bromomethyl)propane-1,3-diol (20 g), bromoacetic acid (42.44 g), p-toluenesulfonic acid monohydrate (1.23 g) and toluene (250 mL). The mixture was heated to reflux under agitation and kept at 114° C. for 4.5 hours, during which water was removed and collected via a Dean-Stark apparatus. The reaction was monitored by gas chromatography (GC). After reaction was complete as indicated by GC, the reaction mixture was cooled to room temperature, washed several times with saturated aqueous sodium bicarbonate (5×150 mL) and phase separated. The organic layer was dried over MgSO₄ and filtered. After filtration, toluene was removed from the organic layer using a rotary evaporator, and then the organic layer was further dried under high vacuum (˜1 torr) to obtain 33.38 g of product as a clear liquid.

Components referred to by chemical name or formula anywhere in the specification or claims hereof, whether referred to in the singular or plural, are identified as they exist prior to coming into contact with another substance referred to by chemical name or chemical type (e.g., another component, a solvent, or etc.). It matters not what chemical changes, transformations and/or reactions, if any, take place in the resulting mixture or solution as such changes, transformations, and/or reactions are the natural result of bringing the specified components together under the conditions called for pursuant to this disclosure. Thus the components are identified as ingredients to be brought together in connection with performing a desired operation or in forming a desired composition. Also, even though the claims hereinafter may refer to substances, components and/or ingredients in the present tense (“comprises”, “is”, etc.), the reference is to the substance, component or ingredient as it existed at the time just before it was first contacted, blended or mixed with one or more other substances, components and/or ingredients in accordance with the present disclosure. The fact that a substance, component or ingredient may have lost its original identity through a chemical reaction or transformation during the course of contacting, blending or mixing operations, if conducted in accordance with this disclosure and with ordinary skill of a chemist, is thus of no practical concern.

The invention may comprise, consist, or consist essentially of the materials and/or procedures recited herein.

As used herein, the term “about” modifying the quantity of an ingredient in the compositions of the invention or employed in the methods of the invention refers to variation in the numerical quantity that can occur, for example, through typical measuring and liquid handling procedures used for making concentrates or use solutions in the real world; through inadvertent error in these procedures; through differences in the manufacture, source, or purity of the ingredients employed to make the compositions or carry out the methods; and the like. The term about also encompasses amounts that differ due to different equilibrium conditions for a composition resulting from a particular initial mixture. Whether or not modified by the term “about”, the claims include equivalents to the quantities.

Except as may be expressly otherwise indicated, the article “a” or “an” if and as used herein is not intended to limit, and should not be construed as limiting, the description or a claim to a single element to which the article refers. Rather, the article “a” or “an” if and as used herein is intended to cover one or more such elements, unless the text expressly indicates otherwise.

This invention is susceptible to considerable variation in its practice. Therefore the foregoing description is not intended to limit, and should not be construed as limiting, the invention to the particular exemplifications presented hereinabove.

1. A nonaqueous electrolyte solution for a lithium battery, which solution comprises i) a liquid electrolyte medium; ii) a lithium-containing salt; and iii) at least one oxygen-containing brominated flame retardant selected from
a) a brominated monoester
α) which has three carbon atoms and at least one bromine atom, or
β) which has at least four carbon atoms and at least one bromine atom, and is represented by the formula 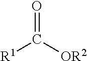
wherein
R¹ and R² each have at least one carbon atom, and at least one of R¹ and R² has at least one bromine atom, with the proviso that when R² is a benzyl group and there is at least one bromine atom in the benzyl group, R¹ is a branched or straight chain alkyl group; and
b) a brominated diester which has at least six carbon atoms and at least one bromine atom, and is represented by the formula 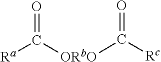
wherein
R^{a}, R^{b}, and R^{c} each have at least one carbon atom, and at least one of R^{a}, R^{b}, and R^{c} has at least one bromine atom.

2. A solution as in claim 1 wherein the oxygen-containing brominated flame retardant is
I) a) a brominated monoester which has four to about twelve carbon atoms and/or one to about eight bromine atoms; or
b) a brominated diester which has six to about twenty carbon atoms and/or one to about eight bromine atoms; or
II) a) a brominated monoester in which R¹ has at least one bromine atom and one to about eight carbon atoms; or
b) a brominated diester which has about six to about eighteen carbon atoms and/or one to about six bromine atoms.

3. (canceled)

4. A solution as in claim 1 wherein the oxygen-containing brominated flame retardant is
I) a) a brominated monoester in which R² has at least one bromine atom and two to about eight carbon atoms; or
b) a brominated diester in which R^{b} has at least one bromine atom and/or R^{a} and R^{c} are the same, or
II) a) a brominated monoester in which R¹ and R² each have at least one bromine atom, R¹ has one to about eight carbon atoms, and R² has one to about nine carbon atoms; or
b) a brominated diester in which at least one bromine atom is present in each of R^{a}, R^{b}, and R^{c}.

5. (canceled)

6. A solution as in claim 1 wherein the oxygen-containing brominated flame retardant is
a) a brominated monoester in which R² does not contain quaternary carbon atoms; or
b) a brominated diester in which R^{b} does not contain quaternary carbon atoms.

7. A solution as in claim 1 wherein the brominated monoester is 2-bromoethyl acetate, 4-bromobutyl acetate, 2-bromovinyl acetate, 3-bromoallyl acetate, 3-bromoallyl butyrate, 2,3-dibromoprop-2-en-1-yl acetate, 2,3-dibromoprop-2-en-1-yl propanoate, (3,5-dibromophenyl)methyl acetate, 3-bromo-2,2-bis(bromomethyl)propyl acetate, methyl 3-bromo-2-propenoate, methyl 2-bromo-acrylate, 2,3-dibromoprop-2-en-1-yl bromoacetate, or (3,5-dibromophenyl)methyl bromoacetate.

8. A solution as in claim 1 wherein two to about four carbon atoms of R^{b} form a bridge between the two ester moieties in the brominated diester.

9. A solution as in claim 1 wherein the brominated diester is 2,3-dibromobut-2-ene-1,4-diyl diacetate, 2,3-dibromobut-2-ene-1,4-diyl bis(2-methylpropanoate), 2,3-dibromobut-2-ene-1,4-diyl bis(2-bromobutanoate), 2,2-bis(bromomethyl)-1,3-propanediyl diacetate, or 2,2-bis(bromomethyl)propane-1,3-diyl bis(bromoacetate).

10. A solution as in claim wherein the oxygen-containing brominated flame retardant is in an amount of about 9.5 wt % or more bromine relative to the total weight of the solution.

11. A solution as in claim 1 wherein the liquid electrolyte medium is ethylene carbonate, ethyl methyl carbonate, or a mixture thereof, and/or wherein the lithium-containing salt is lithium hexafluorophosphate, lithium di(fluoro)(oxalato)borate, or lithium bis(oxalato)borate.

12. A solution as in claim 1 further comprising at least one electrochemical additive selected from:
I)
a) unsaturated cyclic carbonates containing three to about six carbon atoms,
b) fluorine-containing saturated cyclic carbonates containing three to about five carbon atoms and one to about four fluorine atoms,
c) tris(trihydrocarbylsilyl) phosphites containing three to about nine carbon atoms,
d) trihydrocarbyl phosphates containing three to about twelve carbon atoms,
e) cyclic sultones containing three to about eight carbon atoms,
f) saturated cyclic hydrocarbyl sulfites having a 5-membered or 6-membered ring and containing two to about six carbon atoms,
g) saturated cyclic hydrocarbyl sulfates having a 5-membered or 6-membered ring and containing two to about six carbon atoms,
h) cyclic dioxadithio polyoxide compounds having a 6-membered, 7-membered, or 8-membered ring and containing two to about six carbon atoms,
i) another lithium-containing salt, and
j) mixtures of any two or more of the foregoing; or
II)
a) unsaturated cyclic carbonates containing three to about four carbon atoms,
b) fluorine-containing saturated cyclic carbonates containing three to about four carbon atoms and one to about two fluorine atoms,
c) tris(trihydrocarbylsilyl) phosphites containing three to about six carbon atoms,
d) trihydrocarbyl phosphates containing three to about nine carbon atoms,
e) cyclic sultones containing three to about four carbon atoms,
f) saturated cyclic hydrocarbyl sulfites having a 5-membered ring and containing two to about four carbon atoms,
g) saturated cyclic hydrocarbyl sulfates having a 5-membered ring and containing two to about four carbon atoms,
h) cyclic dioxadithio polyoxide compounds having a 6-membered or 7-membered ring and containing two to about four carbon atoms,
i) another lithium-containing salt, and
j) mixtures of any two or more of the foregoing.

13. (canceled)

14. A solution as in claim 12 wherein the electrochemical additive is selected from:
a) an unsaturated cyclic carbonate in an amount of about 0.5 wt % to about 12 wt %, relative to the total weight of the nonaqueous electrolyte solution,
b) a fluorine-containing saturated cyclic carbonate in an amount of about 0.5 wt % to about 8 wt %, relative to the total weight of the nonaqueous electrolyte solution,
c) a tris(trihydrocarbylsilyl) phosphite in an amount of about 0.1 wt % to about 5 wt %, relative to the total weight of the nonaqueous electrolyte solution,
d) a trihydrocarbyl phosphate in an amount of about 0.5 wt % to about 5 wt %, relative to the total weight of the nonaqueous electrolyte solution,
e) a cyclic sultone in an amount of about 0.25 wt % to about 5 wt %, relative to the total weight of the nonaqueous electrolyte solution,
f) a saturated cyclic hydrocarbyl sulfite in an amount of about 0.5 wt % to about 5 wt %, relative to the total weight of the nonaqueous electrolyte solution,
g) a saturated cyclic hydrocarbyl sulfate in an amount of about 0.25 wt % to about 5 wt %, relative to the total weight of the nonaqueous electrolyte solution,
h) a cyclic dioxadithio polyoxide compound in an amount of about 0.5 wt % to about 5 wt %, relative to the total weight of the nonaqueous electrolyte solution,
i) another lithium-containing salt in an amount of about 0.5 wt % to about 5 wt %, relative to the total weight of the nonaqueous electrolyte solution, and
j) mixtures of any two or more of the foregoing.

15. A solution as in claim 12 wherein each electrochemical additive is not used with other electrochemical additives.

16. A solution as in claim 1 wherein the solution further comprises a nitrile compound, optionally wherein the nitrile compound is succinonitrile, or wherein the solution further comprises a nitrile compound and another lithium-containing salt, optionally wherein the nitrile compound is succinonitrile and the lithium-containing salt is lithium di(fluoro)(oxalato)borate.

17-19. (canceled)

20. A nonaqueous lithium battery comprising a positive electrode, a negative electrode, and a nonaqueous electrolyte solution as in claim 1.

21. A nonaqueous electrolyte solution for a lithium battery as in claim 1, in which the
at least one oxygen-containing brominated flame retardant is selected from the group consisting of methyl bromoacetate, 2-bromoethyl acetate, 4-bromobutyl acetate, 2-bromovinyl acetate, 3-bromoallyl acetate, 3-bromoallyl butyrate, 2,3-dibromoprop-2-en-1-yl acetate, 2,3-dibromoprop-2-en-1-yl propanoate, (3,5-dibromophenyl)methyl acetate, 3-bromo-2,2-bis(bromomethyl)propyl acetate, methyl 3-bromo-2-propenoate, methyl 2-bromo-acrylate, 2,3-dibromoprop-2-en-1-yl bromoacetate, 2,3-dibromoprop-2-en-1-yl 2-methylpropanoate, (3,5-dibromophenyl)methyl bromoacetate, 2,3-dibromobut-2-ene-1,4-diyl diacetate, 2,3-dibromobut-2-ene-1,4-diyl bis(2-methylpropanoate), 2,3-dibromobut-2-ene-1,4-diyl bis(2-bromobutanoate), 2,2-bis(bromomethyl)-1,3-propanediyl diacetate, and 2,2-bis(bromomethyl)propane-1,3-diyl bis(bromoacetate).

22. A solution as in claim 21 wherein the oxygen-containing brominated flame retardant is in an amount of about 9.5 wt % or more bromine relative to the total weight of the solution.

23. A solution as in claim 21 wherein the liquid electrolyte medium is ethylene carbonate, ethyl methyl carbonate, or a mixture thereof, and/or wherein the lithium-containing salt is lithium hexafluorophosphate, lithium di(fluoro)(oxalato)borate, or lithium bis(oxalato)borate.

24. A solution as in claim 21 wherein the solution further comprises a nitrile compound, optionally wherein the nitrile compound is succinonitrile; or a nitrile compound and another lithium-containing salt, optionally wherein the nitrile compound is succinonitrile and the lithium-containing salt is lithium di(fluoro)(oxalato)borate or a nitrile compound and another lithium-containing salt.

25. (canceled)

26. A nonaqueous lithium battery comprising a positive electrode, a negative electrode, and a nonaqueous electrolyte solution as in claim 21.

27. A process for producing a nonaqueous electrolyte solution for a lithium battery, which process comprises combining components comprising i) a liquid electrolyte medium; ii) a lithium-containing salt; and iii) at least one oxygen-containing brominated flame retardant selected from
a) a brominated monoester
α) which has three carbon atoms and at least one bromine atom, or
β) which has at least four carbon atoms and at least one bromine atom, and is represented by the formula 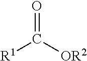
wherein
R¹ and R² each have at least one carbon atom, and at least one of R¹ and R² has at least one bromine atom, with the proviso that when R² is a benzyl group and there is at least one bromine atom in the benzyl group, R¹ is a branched or straight chain alkyl group; and
b) a brominated diester which has at least six carbon atoms and at least one bromine atom, and is represented by the formula 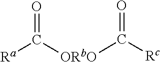
wherein
R^{a}, R^{b}, and R^{c} each have at least one carbon atom, and at least one of R^{a}, R^{b}, and R^{c} has at least one bromine atom.

28. A process as in claim 27 wherein the components further comprise at least one electrochemical additive selected from:
a) unsaturated cyclic carbonates containing three to about six carbon atoms,
b) fluorine-containing saturated cyclic carbonates containing three to about five carbon atoms and one to about four fluorine atoms,
c) tris(trihydrocarbylsilyl) phosphites containing three to about nine carbon atoms,
d) trihydrocarbyl phosphates containing three to about twelve carbon atoms,
e) cyclic sultones containing three to about eight carbon atoms,
f) saturated cyclic hydrocarbyl sulfites having a 5-membered or 6-membered ring and containing two to about six carbon atoms,
g) saturated cyclic hydrocarbyl sulfates having a 5-membered or 6-membered ring and containing two to about six carbon atoms,
h) cyclic dioxadithio polyoxide compounds having a 6-membered, 7-membered, or 8-membered ring and containing two to about six carbon atoms,
i) another lithium-containing salt, and
j) mixtures of any two or more of the foregoing.

29. A process for producing a nonaqueous electrolyte solution for a lithium battery as in claim 27, in which the
at least one oxygen-containing brominated flame retardant is selected from the group consisting of methyl bromoacetate, 2-bromoethyl acetate, 4-bromobutyl acetate, 2-bromovinyl acetate, 3-bromoallyl acetate, 3-bromoallyl butyrate, 2,3-dibromoprop-2-en-1-yl acetate, 2,3-dibromoprop-2-en-1-yl propanoate, (3,5-dibromophenyl)methyl acetate, 3-bromo-2,2-bis(bromomethyl)propyl acetate, methyl 3-bromo-2-propenoate, methyl 2-bromo-acrylate, 2,3-dibromoprop-2-en-1-yl bromoacetate, 2,3-dibromoprop-2-en-1-yl 2-methylpropanoate, (3,5-dibromophenyl)methyl bromoacetate, 2,3-dibromobut-2-ene-1,4-diyl diacetate, 2,3-dibromobut-2-ene-1,4-diyl bis(2-methylpropanoate), 2,3-dibromobut-2-ene-1,4-diyl bis(2-bromobutanoate), 2,2-bis(bromomethyl)-1,3-propanediyl diacetate, and 2,2-bis(bromomethyl)propane-1,3-diyl bis(bromoacetate).

30. A process as in claim 29 wherein the components further comprise at least one electrochemical additive selected from vinylene carbonate, 4-fluoro-ethylene carbonate, tris(trimethylsilyl)phosphite, triallyl phosphate, 1,3-propane sultone, 1,3-propene sultone, ethylene sulfite, 1,3,2-dioxathiolane 2,2-dioxide, 1,5,2,4-dioxadithiane 2,2,4,4-tetroxide, lithium di(fluoro)(oxalato)borate, lithium bis(oxalato)borate, and mixtures of any two or more of these.

31. Each of the following molecules separately, as a new composition of matter:
2,3-dibromoprop-2-en-1-yl propanoate;
2,3-dibromoprop-2-en-1-yl 2-methylpropanoate;
2,3-dibromoprop-2-en-1-yl bromoacetate;
2,3-dibromobut-2-ene-1,4-diyl bis(2-methylpropanoate);
2,3-dibromobut-2-ene-1,4-diyl bis(2-bromobutanoate).
